(19) United States
(12) Patent Application Publication
(10) Pub. No.: US 2007/0015191 A1
(43) Pub. Date: Jan. 18, 2007

(54) Network of buoyant particles for biomolecule purification and use of buoyant particles or network of buoyant particles for biomolecule purification

(75) Inventors: Rex M. Bitner, Cedarburg, WI (US); Michelle Mandrekar, Oregon, WI (US); Don B. Smith, Evansville, WI (US); Douglas H. White, Madison, WI (US)

Correspondence Address: FITZPATRICK CELLA HARPER & SCINTO, 30 ROCKEFELLER PLAZA, NEW YORK, NY, 10112, US

(73) Assignee: Promega Corporation, Madison, WI (US)

(21) Appl. No.: 11/477,491

(22) Filed: Jun. 30, 2006

Related U.S. Application Data
(60) Provisional application No. 60/695,545, filed on Jul. 1, 2005.

(51) Int. Cl. C12Q 1/68

(52) U.S. Cl. 435/6

(57) ABSTRACT

A network of buoyant particles for clearing lysates of biological material, the network including two or more buoyant particles covalently linked together, wherein the network ranges in size from approximately 30 microns to approximately one centimeter along the network's longest dimension. The buoyant particles may have a silica surface. The network may have a density less than about 1.2 g/cm³. Methods of making the network of buoyant particles and methods of isolating target biological material using buoyant particles or a network of buoyant particles are also described.

This application claims the benefit of U.S. Provisional Application No. 60/695,545, which was filed Jul. 1, 2005.

### FIELD OF THE INVENTION

This invention relates to biomolecule purification and methods and kits for biomolecule purification. In particular, this invention relates to a network of buoyant particles used for biomolecule purification. Specifically, buoyant particles are covalently linked together to form a network of buoyant particles. This invention also relates to methods and kits using buoyant particles or a network of buoyant particles for biomolecule purification. In particular, this invention relates to using the buoyant particles or a network of buoyant particles for separating a target biomolecule from solutions of disrupted biological material, such as lysates or homogenates of bacteria, plant tissue or animal tissue.

### BACKGROUND OF THE INVENTION

Biomolecule purification is a key step for many applications in molecular biology. Accordingly, a variety of components and methods have been developed to efficiently isolate target biological material with a high yield. For example, U.S. Pat. No. 6,027,945 (Smith et al.) discloses methods of isolating biological target materials using silica magnetic particles. The Smith et al. patent discloses methods involving forming a complex of silica magnetic particles and the target biological material in a medium and separating the target biological material using magnetic force.

Additionally, U.S. Pat. No. 6,787,307 B1 (Bitner et al.), which is hereby incorporated by reference in its entirety, discloses lysate clearance and nucleic acid isolation using silanized silica matricies. The Bitner et al. patent discloses that silanized silica matricies may be used to isolate plasmid DNA, fragments of DNA, chromosomal DNA, or RNA from various contaminants such as proteins, lipids, cellular debris, or non-target nucleic acids. The silanized silica matricies include a silica based solid phase and a plurality of silane ligands covalently attached to the surface of the solid phase. The solid phase includes silica, preferably in the form of silica gel, siliceous oxide, solid silica such as glass fiber, glass beads, or diatomaceous earth, or a mixture of two or more of the above.

Despite these advancements, a need still exists in the art to enhance yields of isolated biological material, particularly in methods involving filtration and/or centrifugation. This invention is directed toward remedying this problem.

### SUMMARY OF THE PRESENT INVENTION

Generally, the present invention is directed to a network of buoyant particles, and the use of buoyant particles and a network of buoyant particles in biomolecule purification.

In one aspect, a network of buoyant particles for clearing lysates of biological material comprises two or more buoyant particles covalently linked together, wherein the network ranges in size from approximately 30 microns to approximately one centimeter along the network's longest dimension.

Preferably, the buoyant particles have a silica or a silica-containing surface. Also preferably, the buoyant particles or the network of buoyant particles have a density less than about 1.2 g/cm³.

Preferably, the network ranges in size from approximately 30 microns to approximately 1 mm along the network's longest dimension. More preferably, the network ranges in size from approximately 100 microns to approximately 500 microns along the network's longest dimension.

In a second aspect, the invention is directed toward a method of making a network of buoyant particles for clearing lysates of biological material. The method includes the steps of: (a) placing buoyant particles in an alkaline solution containing SiO₂, and (b) adding acid to the solution so that the SiO₂ condenses, covalently linking the buoyant particles together to form the network of buoyant particles.

Preferably, the buoyant particles have a silica or a silica-containing surface. The silica-containing surface may incorporate other elements or compounds such as borate, alumina, zeolite, zirconia or fluorine, but are not limited thereto. Also preferably, the buoyant particles or the network of buoyant particles have a density less than about 1.2 g/cm³.

Preferably, the network ranges in size from approximately 100 microns to approximately 1 mm along the network's longest dimension. More preferably, the network ranges in size from approximately 100 microns to approximately 500 microns along the network's longest dimension.

In a third aspect, the invention is directed toward a method of making a network of buoyant particles for clearing lysates of biological material. The method includes the steps of: (a) placing buoyant particles having a silica or a silica-containing surface in an alkaline solution, and (b) combining the result of step (a) with a salt plus acid solution.

Preferably, the buoyant particles have a silica or a silica-containing surface. Also preferably, the buoyant particles or the network of buoyant particles have a density less than about 1.2 g/cm³.

Preferably, the network ranges in size from approximately 30 microns to approximately 1 mm along the network's longest dimension. More preferably, the network ranges in size from approximately 100 microns to approximately 500 microns along the network's longest dimension.

In a fourth aspect, the invention is directed to a method of isolating target biological material using buoyant particles or a network of buoyant particles for clearing lysates of biological material. The method includes the steps of: (a) adding buoyant particles or network buoyant particles to the biological material; (b) adding a binding solution; (c) performing cell lysis; and (d) performing gravitational, centrifugal, vacuum or positive pressure filtration clearing of non-target biological material that has become associated with the buoyant particles or the network of buoyant particles. The binding solution is added at a concentration sufficient to promote selective adsorption of the target or non-target biological material to the network. In certain embodiments of the invention, the binding solution and cell lysis solution are the same.

Preferably, the binding solution contains at least one of a chaotrope and an alcohol.

Preferably, the method also includes a step of purifying the target biological material. Preferably, the biological material is at least one of bacteria, plant tissue, animal tissue or animal body fluids. Also preferably, the method includes a step of heating the solution after performing cell lysis.

Preferably, the buoyant particles have a silica or a silica-containing surface. Also preferably, the buoyant particles or the network of buoyant particles have a density less than about 1.2 g/cm³.

Preferably, the network ranges in size from approximately 30 microns to approximately 1 mm along the network's longest dimension. More preferably, the network ranges in size from approximately 100 microns to approximately 500 microns along the network's longest dimension.

In a fifth aspect, the invention is directed to a method of isolating target biological material using buoyant particles or a network of buoyant particles for clearing lysates of biological material. The method includes the steps of: (a) combining the buoyant particles or the network of buoyant particles with lysed biological material; and (b) performing gravitational, centrifugal, vacuum filtration or positive pressure filtration clearing of non-target biological material that has become associated with the buoyant particles or the network of buoyant particles.

Preferably, the buoyant particles or network of buoyant particles may be supplied in combination with a lysis solution or a binding solution that promotes the binding of target or non-target biological material with the buoyant particles or the network of buoyant particles. Preferably, the method also includes a step of purifying the target biological material. Also preferably, the biological material is at least one of bacteria, plant tissue, animal tissue, or animal body fluids. Also preferably, the method includes a step of heating the solution prior to performing gravitational, centrifugal, vacuum filtration or positive pressure filtration clearing.

Preferably, the buoyant particles have a silica or a silica-containing surface. Also preferably, the buoyant particles or the network of buoyant particles have a density less than about 1.2 g/cm³.

Preferably, the network ranges in size from approximately 30 microns to approximately 1 mm along the network's longest dimension. More preferably, the network ranges in size from approximately 100 microns to approximately 500 microns along the network's longest dimension.

In a sixth aspect, the invention is directed to a kit for clearing lysates of biological material. The kit includes a container containing a lysis solution and at least one member selected from the group consisting of a buoyant particle, a network of buoyant particles, and a buoyant particle and a network of buoyant particles. Alternatively, the kit includes a first container containing buoyant particles or a network of buoyant particles, and a second container containing a lysis solution.

Preferably, the buoyant particles have a silica or a silica-containing surface. Also preferably, the buoyant particles or the network of buoyant particles have a density less than about 1.2 g/cm³.

Preferably, the network ranges in size from approximately 30 microns to approximately 1 mm along the network's longest dimension. More preferably, the network ranges in size from approximately 100 microns to approximately 500 microns along the network's longest dimension.

A better understanding of these and other features and advantages of the present invention may be had by reference to the accompanying description and Examples, in which preferred embodiments of the invention are illustrated and described.

### DETAILED DESCRIPTION OF THE PREFERRED EMBODIMENTS

The present invention is advantageous in that it can increase the effective yield of target biomolecules to be purified. The effective yield is increased because the buoyant particles or the network of buoyant particles can help reduce filter clogging during a filtration (particularly vacuum filtration or positive pressure filtration) step in a purification process. The buoyant particles or the network of buoyant particles can also help improve the yield of a target biomolecule during a centrifugation step in a purification process because the buoyant particles or the network of buoyant particles can serve as a filter through which a solution containing the target biological material and various contaminants passes during centrifugation.

Accordingly, the methods for using the buoyant particles and the network buoyant particles of this invention have broad utility and can be used, for example, for lysate clearing, plasmid purification, genomic DNA separation from plasmid DNA, and genomic DNA separation from RNA. Of course, the methods are not limited thereto. For each use, the buoyant particles or the network of buoyant particles perform the function of filtering biological material from solution. The filtering function can differ depending on the purification procedure. For example, in some purification methods, it is preferable to have non-target biological material associate with the buoyant particles or the network of buoyant particles, allowing the target biological material to pass through and remain in solution. In other purification methods, it is preferable for the target biological material to bind to the buoyant particles or the network buoyant particles, allowing non-target biological material to pass through and remain in solution.

Use of a network of buoyant particles to filter a solution of disrupted biological material is also advantageous due to a “rafting” effect of the network buoyant particles in a solution. This “rafting” effect occurs because the hydrodynamic drag of rising in solution of the network of buoyant particles is reduced as compared to individual buoyant particles. The reduced hydrodynamic drag allows the network of buoyant particles to float on the solution, preventing other cellular debris from clogging the filter during a filtration or centrifugation step of a purification process.

To create a network of buoyant particles, individual buoyant particles are covalently linked together. For example, the network of buoyant particles may be formed by coating buoyant particles with silica or a composition containing silica and then fusing the particles together through a condensation reaction. Of course, if the buoyant particles already have a silica surface, the particles may be covalently linked together without adding additional silica. The types of buoyant particles suitable for this invention are not particularly limited. Examples of preferable buoyant particles include polyurethane particles, polyvinylidene difluoride particles, high density polyethylene particles, Scotchlite™ S60/10,000 and H50/10,000 glass bubbles (3M Company, St. Paul, Minn., USA), but the invention is not limited thereto.

In addition, depending on the particular function to be performed by the buoyant particles or the network of buoyant particles, the surface of the buoyant particles may be modified. The modification may occur prior to the formation of the network of buoyant particles, or alternatively, the surface of the network of buoyant particles may be modified after the network has been formed. For instance, the buoyant particles may be silanized, and a method of making silanized buoyant particles is described in the Examples below.

Regardless of the method of manufacture and surface treatment, the network of buoyant particles of this invention includes two or more buoyant particles covalently linked together. The resulting network ranges in size from approximately 30 microns to approximately one centimeter along the network's longest dimension. Preferably, the network ranges in size from approximately 100 microns to approximately 1 mm along the network's longest dimension. More preferably, the network ranges in size from approximately 100 microns to approximately 500 microns along the network's longest dimension. Moreover, the network of buoyant particles preferably has a density less than about 1.2 g/cm³. More preferably, the network of buoyant particles has a density between 0.5 and 0.8 g/cm³.

As noted above, the buoyant particles and the network of buoyant particles may be used to clear lysates of biological material. In one approach, the particles or the network is designed such that the target biological material does not bind to the buoyant particles or the network of buoyant particles. In such a scenario, for example, the buoyant particles or the network buoyant particles first may be added to a container of biological material. Cell lysis is then performed. A binding solution is then added at a concentration sufficient to promote the selective adsorption of the disrupted biological material. It should be noted that the binding solution may be added either before or after cell lysis. Additionally, it should be noted that one solution may perform as both the binding solution and the cell lysis solution. The disrupted contents of the cells come into contact with the buoyant particles or the network of buoyant particles. Since the non-target material has an affinity for the buoyant particles or the network of buoyant particles, the non-target material forms a complex with the buoyant particles or the network of buoyant particles. Then, the non-target biological material that has become associated with the buoyant particles or the network of buoyant particles is cleared via a gravitational, centrifugal, vacuum filtration or positive pressure filtration clearing step. The above steps may be repeated as desired to increase the yield of the target biological material. Of course, the method may also be modified by performing cell lysis prior to the addition of the buoyant particles or the network of buoyant particles, and the method may be modified so that the target biological material is selectively adsorbed to the buoyant particles or the network of buoyant particles. If a magnetic purification step is used, a solution containing magnetic particles, such as MagneSil® Paramagnetic Particles (Promega Corp., Madison, Wis.), needs to be added to the solution containing the biological material.

The binding solution used in the above-described method preferably contains a chaotrope, an alcohol, or mixtures thereof. The presence of the chaotrope, alcohol, or mixture thereof facilitates the adsorption of the biological material to the buoyant particles or network of buoyant particles.

It should be noted, too, that the methodologies of the present invention are not limited to the use of one type of buoyant particle or the use of one network of buoyant particles. Rather, the methodologies may include the use of two or more types of buoyant particles, or the use of buoyant particles in combination with a network of buoyant particles. The methodologies may also include use of two or more types of networks of different buoyant particles. The selection of buoyant particle(s) and/or network(s) of buoyant particles depends on the particular application for which the particle(s) and/or network(s) are to be used. In addition, the particles and/or networks may be used together or sequentially.

To further enhance the effective yield of the target biological material, a step of heating the lysis solution may be added to the above-described methods. Heating the lysis solution increases the efficiency of the cell lysis, which helps to improve the yield of the target biological material. For an example demonstrating the effect of heating the lysis solution on the yield of the target biological material, see Example 11, below.

In another aspect of the present invention, the buoyant particles or network buoyant particles may be packaged in a kit. One typical kit includes a container of the buoyant particles or the network buoyant particles and a container of lysis solution. Another kit may include a container of a first type of buoyant particles, a container of a second type of buoyant particles, as well as a container of lysis solution. Additionally, a kit may include a container of network buoyant particles, a container of buoyant particles, and a container of lysis solution. In fact, depending on the particular application for which the kit is to be used, the kit may include any combination of types of buoyant particles and/or types of networks of buoyant particles. Alternatively, a kit may include a container containing a lysis solution and at least one member selected from the group consisting of a buoyant particle, a network of buoyant particles, and a buoyant particle and a network of buoyant particles. The kits may additionally include a clearing column, or the like. The clearing column helps to separate target biological material from non-target biological material.

One of ordinary skill in the art of the present invention will be able to use the present disclosure to select other buoyant particles than those used in this disclosure to illustrate the principles of the invention.

The Examples of this disclosure should not limit the scope of the present invention. Modifications to the present invention will be apparent to those of skill in the art.

### EXAMPLES

### Example 1

### Making Network Buoyant Particles with the Addition of SiO₂ by Batch Synthesis in a Vessel

Into a 50 ml plastic screw-cap tube, 4.55 gm of silicic acid was added to 5.2 ml of 56% KOH (weight/volume). Water was added to give a final volume of 50 ml. The tube was then incubated in 50° C. water with occasional stirring to facilitate solubilization of the solution.

Ten milliliters of this solution was added to a 50 ml screw-cap tube containing 7.5 grams of S60/10,000 glass bubbles as buoyant particles. The tube was inverted several times to resuspend the glass bubbles in the solution. The tube was left capped and inverted (screw-cap side down) to allow the glass bubbles to float upward under 1×gravity. After 20 minutes, the tube was gently inverted and the liquid pipetted off (about 8.8 ml of the initial 10 ml of solution was removed). Then, 7.5 ml of 5.0 M HCl was added to the tube, and the tube was gently mixed. The addition of the HCl covalently linked the SiO₂ coated glass bubbles into clumps of networks of buoyant particles through a condensation reaction.

The mixture of networks of buoyant particles was pipetted up into a 10 ml plastic pipet, and the pipet was left in a vertical position (tip down) for 20 minutes. After 20 minutes, the networks of buoyant particles had floated to the top, and the HCl solution in the bottom of the pipet was removed and discarded. A solution of water was pipetted up into the pipet, then the mixture was pipetted out into a fresh 50 ml tube and gently mixed by several pipettings up and down. The solution was then drawn up into the pipet and the pipet was left in a vertical position (tip down) for 20 minutes. This process was repeated for a total of five water washes. After the fifth wash, the wash was discarded and a solution of 260 mM KOAc pH 4.8 was used to resuspend the networks of buoyant particles and neutralize the pH of the solution. The networks of buoyant particles were then washed one more time with water, using the above method.

### Example 2

### Making a Network of Buoyant Particles with the Addition of SiO₂ by Column Synthesis

Initially, S60/10,000 glass bubbles, as buoyant particles, were stirred into a water solution in a beaker so that intact bubbles would float on the water surface. This allowed the intact bubbles to be separated from broken bubbles and bubble particles, which sink in a water solution.

Twenty-six (26) grams of the floating glass bubbles were placed into a 50 ml plastic tube. Five (5) milliliters of the SiO₂/KOH solution described in Example 1, above, was added to the glass bubbles and mixed thoroughly for 10 minutes at room temperature. The glass bubble suspension was then added to PureYield™ clearing columns (catalog # A2490, Promega Corporation, Madison, Wis., USA), about 14 ml per column. The clearing column membrane retained glass bubble particles, and allowed liquid to pass through. The column capacity of 20 ml allowed for the subsequent addition of HCl solutions to partially filled columns without column overflow.

The clearing columns containing the glass bubbles were allowed to drain under 1×gravity. The glass bubbles were then washed by the addition of 5 ml of 1.0 N HCl to each column. The HCl was allowed to drain from the column. Two additional washes using 5 ml of 1.0 N HCl were similarly performed. At the end of the third HCl application, the effluent at the bottom of the columns was monitored using pH indicator paper to ensure the pH was below pH 2.

The columns were then washed three times, using 7 ml of water per wash, per column. The columns were then washed with 8 ml of 4 M guanidine isothiocyanate/10 mM Tris pH 7.5, and the liquid was allowed to drain at 1×gravity.

### Example 3

### Making a Network of Buoyant Particles without Additional Silica by Column Synthesis Method

Two solutions were prepared for later use in the procedure: (1) “LiCl in HCl” was made by adding 4.24 gm LiCl, 5.0 ml of water and 10 ml of concentrated HCl; and (2) “CaCl₂ in HCl” was made by adding 14.7 gm of CaCl₂, 15 ml of water and 30 ml of concentrated HCl.

Then, 2.0 gm of S60/10,000 glass bubbles, as buoyant particles having a silica surface, were weighed in a 50 ml plastic tube, 6.0 ml of 6% LiOH in water was added, and the contents mixed thoroughly. This tube is “tube Li”. Similarly, 2.0 gm of S60/10,000 glass bubbles were weighed in a second 50 ml plastic tube, 6.0 ml of a saturated solution of Ca(OH)₂ in water was added, and the contents mixed thoroughly. This tube is “tube Ca”.

The suspensions were added to PureYield™ clearing columns (catalog #A246B, Promega Corporation, Madison, Wis., USA) placed in 50 ml plastic tubes, and allowed to settle for 10 minutes at 1×gravity. The tubes were centrifuged for 30 seconds at 500×gravity to allow the solution to flow through the columns, with the S60/10,000 glass bubbles retained in the clearing columns.

Next, 4.0 ml of “LiCl in HCl” (above) was added to tube Li, and 4.0 ml of “CaCl₂ in HCl” (above) was added to tube Ca. Each solution was mixed thoroughly by pipetting. The tubes were allowed to drip at 1×gravity for 60 minutes, then the pH of the ending flow-through solution on the bottom of the clearing column was tested, and each solution was found to be about pH 2 by pH indicator paper. Then 10 ml of water was added to each column, without pipette mixing, and the columns were allowed to drip at 1×gravity for 60 minutes. This step was repeated for a total of 3 washes of 10 ml of water, per column. Then 10 ml of 1.32 M KOAc, pH 4.8, was used to wash each column, similarly with the water washes. The column flow-through at the bottom of each column was found to be about pH 4.8. The particles in each column were then washed with 10 ml of water, as above. Finally, the particles were removed from the clearing columns and placed into clean 50 ml tubes, and dried overnight under vacuum.

### Example 4

### Making Silanized Buoyant Particles

gm of S60/10,000 glass bubbles was resuspended in 20 ml of 95% methanol in a 50 ml plastic screw cap tube. 3.0 ml of 3-glycidoxypropyl trimethoxy silane (Aldrich 44,016-7, St. Louis, Mo., USA) was added. The reaction tube was mixed overnight at room temperature on a platform shaker. Then 10 ml of water was added to increase solution density and the particles were allowed to float to the surface. 30 ml of the solution was pipetted off the bottom of the tube. The particles were then washed with 30 ml of water, and the particles were allowed to float in the tube. 30 ml was removed by pipette from the tube bottom, leaving 10 ml of particles. The particles were again washed with 30 ml of water, and the particles were allowed to float in the tube. 30 ml of solution was removed from the bottom by pipette, for a total of three water washes. The silanized particles were transferred into a clearing column (Promega catalog #246B), which was placed into a 50 ml tube and centrifuged for 5 minutes at 200×gravity. The particles were dried under vacuum (17 inches of mercury) for three hours.

### Example 5

### Comparative DNA Binding Capacity of Buoyant Particles and Networks of Buoyant Particles

0.4 gm of each of the particles shown in Table 1 below, were weighed into a clearing column (Promega cat # 246B) which was placed into a 50 ml screw-cap tube. Four 400 ml JM109 (pGEM) plasmid lysates were prepared as described in the protocol of Example 7, below, for tubes 1 and 2, up to the end of the sentence in paragraph [0068] stating: “The solutions were allowed to sit in the columns for 2 minutes, then the tubes were centrifuged for 10 minutes at 2000×gravity through A246B PureYield™ Clearing Columns, and the flow-through solutions captured in the 50 ml conical tubes.” The 4 tubes of lysate flow-through were pooled into one cleared lysate. To each column containing the particles listed in the Table 1 below, 5 ml of this cleared lysate was added, and the columns were allowed to drip under 1×gravity. The flow-through of each column was reapplied to the particles a total of 5 times to ensure that exposure of the particle surface to the plasmid DNA had reached a level of saturation. The columns were centrifuged at 2000×gravity for 5 minutes, and 10 ml of “no plasmid lysate solution” was added per column. This “no plasmid lysate solution” was made as follows:

Four 50 ml tubes, each containing 12 ml Resuspension Solution plus 12 ml Lysis Solution plus 20 ml of Neutralization Solution (as described in Example 6 below) were mixed and centrifuged at 2000×gravity for 10 minutes. Then 10 ml of “no plasmid lysate solution” was added per column, as described above, to wash away plasmid DNA not bound to the particles, and the columns were centrifuged at 2000×gravity for 5 minutes. Then 10 ml of Column Wash Solution (described in Example 6) was added per column, and the columns were centrifuged for 5 minutes at 2000×gravity. Next, 10 ml of Column Wash Solution was added per column and the columns were centrifuged for 5 minutes at 2000×gravity, for a total of two column washes. The plasmid DNA was eluted in 2.0 ml of Nuclease Free Water, and measured by absorbance at 260 nm. Because the empty clearing column bound 41.5 gm of DNA in the absence of particles, it was necessary to subtract that amount of DNA from the columns containing particles, as shown below. When these particles are used in plasmid preps, debris will occupy a significant amount of the surface area of the particles. Therefore, the DNA binding capacity of the particles would be expected to be reduced when used in plasmid preps similar to those described in Examples 6 and 7, below.

TABLE 1

| | | | | | Total | Total | μg After |
|---|---|---|---|---|---|---|---|
| Sample | A230 | A260 | A280 | A320 | ml | μg | Blank |
| No particle blank | 0.31 | 0.50 | 0.25 | 0.01 | 1.7 | 41.58 | 0.00 |
| H50/10,000 in 25 mM | 0.42 | 0.70 | 0.35 | 0.01 | 1.7 | 58.64 | 17.06 |
| KOAc, pH 4.8/1 mM | | | | | | | |
| EDTA | | | | | | | |
| S60/10,000 in water | 1.03 | 2.00 | 1.06 | 0.10 | 1.7 | 161.64 | 120.06 |
| H50/10,000 in water | 0.37 | 0.60 | 0.31 | 0.01 | 1.7 | 50.18 | 8.59 |
| S60 silanized | 0.50 | 0.90 | 0.45 | 0.01 | 1.7 | 75.60 | 34.02 |
| S60 network, silanized | 0.45 | 0.78 | 0.39 | 0.01 | 1.7 | 65.33 | 23.70 |
| H50 network, not-silanized | 1.03 | 2.14 | 1.07 | 0.02 | 1.7 | 179.83 | 138.25 |
| H50 network, silanized | 0.40 | 0.61 | 0.32 | 0.02 | 1.7 | 50.36 | 8.78 |

### Example 6

### Lysate Clearance of High Copy Plasmid Using Vacuum Based Purification

3M Scotchlite™ H50/10,000 glass bubbles were treated as follows.

Five 50 ml conical screw cap tubes, each containing 25 ml of dry H50/10,000 glass bubbles, and 20 ml of autoclaved deionized water were mixed by inversion overnight at room temperature. All five tubes were pooled together in a 600 ml glass beaker, and then split back out into five 50 ml tubes. After allowing the bubbles to float to the top of each tube, the solution below was removed along with a small amount of glass bubbles that sank rather than floated. The solutions were replaced with the following formulations: Tube A was 25 mM KOAc pH 4.8; Tube B was 25 mM KOAc pH 4.8 1 mM EDTA; Tube C was 4.09 M guanidine hydrochloride, 759 mM KOAc, 2.12 M glacial acetic acid (final pH of 4.2); and Tube D was 25 mM KOAc pH 4.8, identical to tube A. All tubes were mixed by inversion at room temperature for 16 hours. Then the solution of Tube D was removed and replaced with 4.09 M guanidine hydrochloride, 759 mM KOAc, 2.12 M glacial acetic acid (final pH of 4.2). Tubes A-D were mixed by inversion at room temperature for another 24 hours.

50 ml of Luria Broth (LB-Miller) bacterial plasmid culture DH5α (pGEM) was centrifuged into twelve 50 ml conical screw cap centrifuge tubes. This was repeated for a total of five repetitions per tube. The result was 12 tubes, each with 250 ml of bacterial culture pelleted per tube, each pellet representing 490 A600 absorbance units of cells per tube. The tubes were frozen at −20° C. for later plasmid DNA extraction.

Plasmid purification was performed using Promega's (Madison, Wis.) A2495 plasmid midi-plasmid purification system, with the following solution compositions:
Cell Resuspension Solution: 50 mM Tris, 10 mM EDTA, 100 μg/ml Rnase A;
Cell Lysis Solution: 0.2 M Sodium Hydroxide, 1% SDS;
Neutralization Solution: 4.09 M Guanidine Hydrochloride, 759 mM potassium acetate, 2.12 M glacial acetic acid;
Endotoxin Removal Wash: 4.2 M Guanidine Hydrochloride, 40% isopropanol;
Column Wash: 162.8 mM Potassium Acetate, 22.6 mM Tris, 0.109 mM EDTA. To 320 ml add 170 ml of 95% ethanol;
Nuclease Free Water;
A246B PureYield™ Clearing Columns, 100 ea; and
A245B PureYield™ Binding Columns, 100 ea.

To each of the 12 tubes of DH5α (pGEM) above, 6.0 ml of Cell Resuspension solution was added and gently mixed. Then 6.0 ml of Cell Lysis solution was added and gently mixed. Next, 10 ml of Neutralization Solution was added, and gently mixed.

Tubes 1 and 2 were centrifuged for 15 minutes at 7000×gravity through A246B PureYield™ Clearing Columns, and the flow-through solutions were captured in 50 ml conical tubes. For tubes 1 and 2, the solutions were poured directly into A245B PureYield™ Binding Columns and a vacuum was applied as described below. For Tubes 3 and 4, no glass bubbles were added, and the solution was gently mixed by tube inversion. For Tubes 5 and 6, 1 ml of H50/10,000 glass bubbles from Tube A above was added, and gently mixed by tube inversion. For Tubes 7 and 8, 1 ml of H50/10,000 glass bubbles from Tube B above was added, and gently mixed by tube inversion. For Tubes 9 and 10, 1 ml of H50/10,000 glass bubbles from Tube C above was added, and gently mixed by tube inversion. For Tubes 11 and 12, 1 ml of H50/10,000 glass bubbles from Tube D above was added, and gently mixed by tube inversion.

The contents of Tubes 3-12 above were added to separate (A246B) clearing columns. Each clearing column was seated over a (A245B) binding column, and the binding column was inserted into a Vac-Man® Vacuum Manifold (Promega cat #A7231). Each stacked pair of columns was allowed to stand at room temperature for 3 minutes, and then vacuum was applied to the columns until either the liquid passed through the clearing membrane, or the column was clogged for 2 minutes (no further dripping observed). The clearing columns were then discarded, and the binding columns washed sequentially with 5 ml of Endotoxin Removal Wash. Then, after all the previous solution had passed through the binding membrane, 5 ml of Column Wash was added. After all the previous Column Wash solution had passed through the binding membrane, 5 ml of Column Wash was added and the solution was drawn through the binding membrane of the column. Then, the columns were dried under continued vacuum for 10 minutes. Next, each column was placed into a 50 ml tube and each column was eluted with 800 μl of nuclease free water. After standing at room temperature for 2 minutes, each tube was centrifuged for 5 minutes at 2500×gravity.

DNA concentrations and yields were determined by absorbance at A260 and by PicoGreen™ (Invitrogen, Carlsbad, Calif.) analysis.

Results:

TABLE 2

| | | | | | | | A | | | | | |
|---|---|---|---|---|---|---|---|---|---|---|---|---|
| | | | | | | | 260 | Average | | | Average | |
| | | | | | | Volume | Total | of 2 | % of | Picogreen | of 2 | % of |
| Sample | Dilution | A230 | A260 | A280 | A260/A280 | μl | μg | Tubes | Spin | μg | Tubes | Spin |
| tube 1 | 0.1 | 0.8 | 1.8 | 1.0 | 1.9 | 460 | 412.8 | 392.0 | 100.0 | 368.0 | 348.0 | 100.0 |
| tube 2 | 0.1 | 0.6 | 1.5 | 0.8 | 1.9 | 510 | 371.1 | | | 329.0 | | |
| tube 3 | 0.1 | 0.6 | 1.4 | 0.7 | 1.9 | 540 | 370.5 | 395.2 | 100.8 | 342.9 | 362.0 | 104.0 |
| tube 4 | 0.1 | 0.6 | 1.4 | 0.7 | 1.9 | 600 | 420.0 | | | 381.0 | | |
| tube 5 | 0.1 | 0.8 | 1.8 | 1.0 | 1.9 | 520 | 478.5 | 474.8 | 121.1 | 473.2 | 459.0 | 131.9 |
| tube 6 | 0.1 | 0.8 | 1.7 | 0.9 | 1.9 | 540 | 471.2 | | | 445.5 | | |
| tube 7 | 0.1 | 0.9 | 2.1 | 1.1 | 1.9 | 460 | 475.8 | 380.4 | 97.0 | 487.6 | 362.0 | 104.0 |
| tube 8 | 0.1 | 0.5 | 1.1 | 0.6 | 1.9 | 510 | 284.9 | | | 237.2 | | |
| tube 9 | 0.1 | 0.6 | 1.4 | 0.7 | 1.9 | 530 | 362.1 | 408.6 | 104.2 | 386.9 | 417.0 | 119.8 |
| tube 10 | 0.1 | 0.8 | 1.8 | 0.9 | 1.9 | 500 | 455.0 | | | 447.5 | | |
| tube 11 | 0.1 | 1.0 | 2.4 | 1.2 | 1.9 | 460 | 546.4 | 511.5 | 130.5 | 407.1 | 431.0 | 123.9 |
| tube 12 | 0.1 | 0.9 | 2.1 | 1.1 | 2.0 | 450 | 476.5 | | | 454.5 | | |

### Example 7

### High Copy Plasmid JM109 (phmGFP) with Cell Concentration and Lysate Clearance Using Centrifugation Based Purification

First, a solution of 50 ml of 1.0 M NaCl/50% ethanol (volume/volume) was prepared. 5.0 ml of the 1 M NaCl/50% ethanol solution then was added to 1.5 gm of dry particles of 3M Scotchlite™ S60/10,000 glass bubbles; 5.0 ml of the 1 M NaCl/50% ethanol solution was added to 1.5 ml of dry S60/10,000 network glass bubble particles; and 5.0 ml of the 1 M NaCl/50% ethanol solution was added to 1.5 gm of dry Scotchlite™ H50/10,000 glass bubbles. These solutions are used below.

50 ml of Luria Broth (LB-Bertani) bacterial plasmid culture JM109 (phmGFP) was centrifuged into eight 50 ml conical screw cap centrifuge tubes. This was repeated for a total of five repetitions per tube. The result was 8 tubes (tubes A), each with 250 ml of bacterial culture pelleted per tube, each pellet representing 250×1.67 A600 absorbance units of cells, per tube. Similarly, 50 ml of Luria Broth (LB-Bertani) bacterial plasmid culture JM109 (phmGFP) was centrifuged into eight 50 ml conical screw cap centrifuge tubes. This was repeated for a total of four repetitions per tube, each final pellet representing 200×1.67 A600 absorbance units of cells per tube (tubes B). By combining a 200 ml pellet with a 250 ml pellet in the protocol below, the combined cell pellets added together equaled 750 A600 absorbance units. The tubes were frozen at −20° C. for later plasmid DNA extraction.

Plasmid purification was performed using Promega's (Madison, Wis.) A2495 plasmid midi-plasmid purification system, with the following solution compositions:
Cell Resuspension Solution: 50 mM Tris, 10 mM EDTA, 100 μg/ml RNase A;
Cell Lysis Solution: 0.2 M Sodium Hydroxide, 1% SDS;
Neutralization Solution: 4.09 M Guanidine Hydrochloride, 759 mM potassium acetate, 2.12 M glacial acetic acid;
Endotoxin Removal Wash: 4.2 M Guanidine Hydrochloride, 40% isopropanol;
Column Wash: 162.8 mM Potassium Acetate, 22.6 mM Tris, 0.109 mM EDTA. To 320 ml add 170 ml of 95% ethanol;
Nuclease Free Water;
A246B PureYield™ Clearing Columns; and
A245B PureYield™ Binding Columns.

To each of the 8 tubes of JM109 (phmGFP) 200 ml pellets (tubes B) above, 3.0 ml of Cell Resuspension solution was added and mixed by vigorous vortexing. The resuspended bacterial cells were then transferred to each of 8 tubes of JM109 (phmGFP) 250 ml pellets (tubes A). Each tube was vigorously vortexed to resuspend the bacterial cells.

To each of the tubes B above, that previously contained 200 ml of pelleted bacterial cells, 3.0 ml of the following were added:
Tubes 1 and 2: a solution of 1.0 M NaCl/50% ethanol was added;
Tubes 3 and 4: a solution containing S60/10,000 Scotchlite™ bubbles (above) was added;
Tubes 5 and 6: a solution containing S60/10,000 network glass bubble particles (above) was added; and
Tubes 7 and 8: a solution containing H50/10,000 Scotchlite™ glass bubbles (above) was added.

The solution from each of the 8 tubes was then added to the corresponding 8 tubes containing 750 A600 optical density units of JM109 (phmGFP) (tubes A), and vortexed vigorously.

Then 6.0 ml of Cell Lysis solution was added to tubes B, and gently mixed, then the lysate was transferred to its corresponding tube in the tubes A set, and gently mixed. Tubes B were discarded. Next, 9 ml of Neutralization Solution was added per tube, and gently mixed. The contents of each tube were added to an A246B PureYield™ Clearing Column, each of which was contained in a 50 ml conical bottom tube. The solutions were allowed to sit in the columns for 2 minutes, then the tubes were centrifuged for 10 minutes at 2000×gravity through A246B PureYield™ Clearing Columns, and the flow-through solutions captured in the 50 ml conical tubes.

The volume contents per tube were:

Tubes 1, 2=14 ml, 14 ml (both tubes clogged);
Tubes 3, 4=17 ml, 17 ml;
Tubes 5, 6=14 ml, 15 ml; and
Tubes 7, 8=17 ml, 18 ml. None of tubes 3-8 clogged.

The flow-through contents of each tube were added to separate (A245B) binding columns, each contained in a 50 ml tube. The tubes were centrifuged for 10 minutes at 2000×gravity. Each of the binding columns was washed with 5 ml of Endotoxin Removal Wash and centrifuged for 5 minutes at 2000×gravity. Then 5 ml of Column Wash was added and centrifuged for 5 minutes at 2000×gravity. Next, a second wash of 5 ml of Column Wash was added per column/tube. The tubes were centrifuged for 5 minutes at 2000×gravity. Then each column was placed into an appropriately marked 50 ml tube, each column was eluted with 800 μl of nuclease free water. After standing at room temperature for 2 minutes, each column/tube was centrifuged for 5 minutes at 2000×gravity.

DNA concentrations and yields were determined by absorbance at A260 and by PicoGreen™ (Invitrogen, Carlsbad, Calif.) analysis.

Results:

TABLE 3

| | | | | | % control |
|---|---|---|---|---|---|
| | ml | | Average | % control | using |
| Sample | cleared | A260 total μg | of 2 | using A260 | PicoGreen |
| Tube 1 | 14 | 442 | 465.5 | 100 | 100 |
| Tube 2 | 14 | 489 | | | |
| Tube 3 | 17 | 447 | 479.5 | 103 | 114 |
| Tube 4 | 17 | 512 | | | |
| Tube 5 | 14 | 428 | 473 | 102 | 119 |
| Tube 6 | 15 | 518 | | | |
| Tube 7 | 17 | 534 | 515.5 | 111 | 108 |
| Tube 8 | 18 | 497 | | | |

### Example 8

### General Methods for Optimizing Lysate Clearance Using Glass Bubbles

While buoyant particles are directly usable for lysate clearance, the performance of clearing debris without clearing target material can often be optimized through the addition of salts or organic molecules. Without limiting the scope of the invention, the use of molecules such as NaCl or alcohol can provide a framework for such optimization methods. Optimally, the salts or organic molecules are added at a concentration that removes a maximum amount of debris, without removing substantial amounts of the target molecule(s). Using NaCl as an example, the ideal amount is high enough to maximally salt out proteins (for example), but still low enough to not remove target nucleic acids. In the case of ethanol, an optimal amount is sufficient to facilitate precipitation of undesired debris from solution, without the precipitation of target nucleic acids. When using both NaCl and alcohol in combination, it is important to keep concentrations low enough to not precipitate the NaCl out of solution. It is generally useful to test a range of salt or organic concentrations and observe the performance of the lysate clearing in qualitative aspects such as turbidity, color, viscosity, or the ability to pass through filters without clogging. Quantitative measures such as target nucleic acid purity and yield are useful for more narrowly defining optimal conditions. The following example (Example 9) exemplifies using such a qualitative method.

### Example 9

### Qualitative Evaluation of Using NaCl and Ethanol in Lysate Clearing

E. coli strain JM109 (phMGFP) was grown in five Erlenmyer flasks (2 liter volume/each) of LB Miller media for 17 hours at 37° C. by shaking at 300 rpm, 1 liter of LB Miller per flask. Cell density was measured at A600. The cells were centrifuged, and pellets were stored at −20° C. 1200 A600 OD units were used per sample. The cells were resuspended in the following solutions by vortexing:

| | | % Cell Resuspension | | | | | |
|---|---|---|---|---|---|---|---|
| Tube | Volume (ml) | Buffer | % Ethanol | NaCl | | H50 Scotchlite ™ | |
| 1 | 5 | 63.5 | 24 | 0.625 | M | 25 | gm in 40 ml |
| 2 | 4 | 87.5 | 0 | 0.625 | M | 25 | gm in 40 ml |
| 3 | 2.5 | 100.0 | 0 | 0 | | 0 | |
| 4 | 5 | 57.5 | 24 | 1.5 | M | 12.5 | gm in 40 ml |

Once the cells were resuspended, 1 ml of 95% ethanol was added to Tube 2, which was then vortexed. To Tube 3, 2.5 ml of a solution containing 45% ethanol, 0.625M NaCl, and 25 gm/40 ml H50 Scotchlite™ glass bubbles was added, and the tube was vortexed. After this procedure, Tubes 2 and 3 visually appeared to be well resuspended, while Tubes 1 and 4 visually appeared to have incompletely resuspended clumps of cells.

5 ml of Cell Lysis Solution (see Example 6 for all solution formulations) was added to each tube, and the tubes were mixed by gently inverting them 5 times. 10 ml of Neutralization Solution was added per tube, and tubes were mixed by inversion as before. After a 2 minute incubation, the lysates were added to a clearing column, which was placed in a 50 ml Corning tube. The tubes were then centrifuged for 5 minutes at 1500×gravity in an IEC Centra MP4 swinging bucket centrifuge. The solution that passed through the clearing column filter was examined for volume and cloudiness.

| | |
|---|---|
| Tube 1: | 15 ml of lysate, very cloudy |
| Tube 2: | 10 ml of lysate, very cloudy |
| Tube 3: | 14 ml of lysate, slight cloudiness |
| Tube 4: | 8 ml of lysate, very cloudy |

Tubes 1 and 3 were then passed over a second clearing column by centrifugation at 1500×gravity for 5 minutes. Tube 1 remained cloudy, while Tube 3 showed clear lysate.

### Example 10

### Method of Preparation for Hydrolyzed Scotchlite™ H50 Glass Bubbles

3M has modified Scotchlite™ H50 glass bubbles so they contain epoxide groups on the particle surface. 10 gm of Scotchlite™ H50 glass bubbles were suspended in 1 N HCl, pH 2.3 (adjusted using 10 M NaOH) to a final 100 mg/ml concentration. This suspension was vigorously mixed using an orbital shaker at 300 rpm for 16 hrs. The container was allowed 20 minutes at room temperature for phase separation, which allowed the buoyant hydrolyzed glass bubbles to float to the surface. Removal of the aqueous phase and non-buoyant fractions of the glass bubbles was accomplished by gently piercing the buoyant bubble phase with a glass pipette and suctioning out the spent liquid. The glass bubbles were then washed twice with 100 ml of sterile H₂O by swirling the container, then repeating the phase separation and waste removal procedure. 10 ml of 5 M NaCl and 52.6 ml 95% EtOH were added to the glass bubble slurry, then sterile H₂O was added to a final volume of 100 ml. The final formulation was 100 mg/ml glass bubbles/0.5M NaCl/50% EtOH.

### Example 11

### Use of Hydrolyzed Scotchlite™ H50 Glass Bubbles as a Filtration Aid

Cultures of high copy plasmid-containing bacterial strain JM109 (phMGFP) were grown overnight and the culture O.D. measured at 600 nm. Defined cell masses of 500, 1000, 1250, 1500, and 2000 O.D. were prepared in quadruplicate by centrifugation of the appropriate amount of overnight culture. Plasmid purifications were performed using the PureYield™ Plasmid Midiprep System (see Example 6, above) and the following reagent compositions:
Cell Resuspension Solution: 50 mM Tris, 10 mM EDTA, 100 μg/ml RNase A;
Cell Lysis Solution: 0.2 M NaOH, 1% SDS;
Neutralization Solution: 4.09 M Guanidine Hydrochloride, 759 mM Potassium
Acetate, 2.12 M Glacial Acetic Acid;
Endotoxin Removal Wash: 4 M Guanidine Hydrochloride, 40% Isopropanol;
Column Wash Solution: 60 mM Potassium Acetate, 8.3 mM EDTA, 60% EtOH;
A246B PureYield™ Clearing Columns; and
A245B PureYield™ Binding Columns*

*Note: for these experiments, a second identical binding disc was added to each binding column prior to use to increase the binding capacity of the column.

Duplicate cell pellets representing each of the different cell mass O.D.₆₀₀ₛ were resuspended in 6.0 ml of Cell Resuspension Solution and transferred to 50 ml conical tubes. 6.0 ml of Cell Lysis Solution was added to each sample, mixing by inversion for three minutes. To one sample from each duplicate set, 2.0 ml of 100 mg/ml H50 glass bubbles were added, mixing by gentle inversion 10-15 times. 6.0 ml of Neutralization was added to all samples.

Each sample was mixed by rapid inversion, then transferred immediately to Clearing Columns placed in 50 ml conical tubes. Cleared lysates were collected by centrifugation at 3000×gravity for five minutes in a swinging bucket centrifuge. The table below shows the resultant effect on cleared lysate volumes between the duplicate sets with and without bubble addition:

| O.D.₆₀₀ cell mass | Lysate Volume | Lysate Volume |
|---|---|---|
| Processed | Bubbles added | No bubbles added |
| 500 | 18.0 ml | 17.5 ml |
| 1000 | 18.0 ml | 14.5 ml |
| 1250 | 17.5 ml | 12.0 ml |
| 1500 | 17.5 ml | 9.5 ml |
| 2000 | 16.0 ml | 9.0 ml |

Increased Plasmid Recovery as a Reflection of Filtration Efficiency and Lysate Recycling:

Cleared lysates were then transferred to the 2-disc Binding Columns in 50 ml conical tubes and centrifuged for three minutes at 1500×gravity. Flow-throughs from the binding step from each sample were collected and set aside. The binding columns were washed successively using 5 ml Endotoxin Removal Wash, and then 20 ml Column Wash. Each step used centrifugation at 1500×gravity for three minutes. Finally, the empty columns were centrifuged at 3000×gravity for five minutes. Plasmid DNA was eluted by applying 3.0 ml of sterile H₂O followed by centrifugation at 3000×gravity for five minutes. Each binding column was then rinsed using 20 ml of sterile H₂O centrifuged at 3000×gravity for five minutes.

For each sample, flow-throughs from the first binding step were now reloaded into the binding column and the binding, washing, drying, elution, and column-rinsing procedures were repeated in identical fashion. Two subsequent bindings and elutions followed, resulting in a total of four-3.0 ml elutions representing each sample. This was done to ensure that differences in overall yield between the conditions were not simply a reflection of the binding efficiency or column binding capacity. Yield estimations were done by spectrophotometry, and resultant yields for the four-elution sets were combined to reflect the total yield of plasmid DNA. The following results were obtained:

| O.D.₆₀₀ cell mass | Plasmid yield in μg | Lysate Volume |
|---|---|---|
| processed | Bubbles added | No bubbles added |
| 500 | 1933 | 2417 |
| 1000 | 3504 | 3383 |
| 1250 | 4108 | 2900 |
| 1500 | 4350 | 2417 |
| 2000 | 4592 | 3383 |

Increased Lysis Efficiency by Microwave Treatments of Cell Lysis Reactions:

The second set of duplicate cell pellets representing each of the different cell mass O.D.₆₀₀ₛ were resuspended in 6.0 ml of Cell Resuspension Solution and transferred to 50 ml conical tubes. 6.0 ml of Cell Lysis Solution was added to each sample, mixing by inversion for three minutes. All of the sample tubes were then placed in a glass beaker filled with enough water that the liquid/air interface of the cell lysates was below the water level in the beaker. This beaker was placed in a 600 W microwave oven set to high and was microwaved for 40 seconds. Each sample tube was gently mixed by inversion for 20 seconds, then returned to the beaker of water and microwaved for an additional 20 seconds until the monitored temperature of the lysates reached approximately 55° C. All were mixed a final time by gentle inversion for 20 seconds, and then were placed in an ice bath for 15 minutes to cool. To one sample from each duplicate set, 2.0 ml of 100 mg/ml hydrolyzed (Example 10) H50 glass bubbles were added, mixing by gentle inversion 10-15 times. 6.0 ml of Neutralization Solution was added to all samples. Samples were mixed by rapid inversion, then transferred immediately to Clearing Columns placed in 50 ml conical tubes. Cleared lysates were collected by centrifugation at 3000×gravity for five minutes in a swinging bucket centrifuge. The resultant effect on cleared lysate volumes between the duplicate sets with and without bubble addition was obtained:

| O.D.₆₀₀ cell mass | Lysate Volume | Lysate Volume |
|---|---|---|
| processed | Bubbles added | No bubbles added |
| 500 | 18.5 ml | 17.5 ml |
| 1000 | 18.5 ml | 16.5 ml |
| 1250 | 18.0 ml | 17.0 ml |
| 1500 | 18.5 ml | 16.5 ml |
| 2000 | 17.5 ml | 14.5 ml |

Cleared lysates were then transferred to the 2-disc Binding Columns in 50 ml conical tubes and centrifuged for three minutes at 1500×gravity. Flow-throughs from the binding step were collected and set aside. The binding columns were washed successively using 5 ml Endotoxin Removal Wash, then 20 ml Column Wash. Each step used centrifugation at 1500×gravity for three minutes. Finally, the empty columns were centrifuged at 3000×gravity for five minutes. Plasmid DNA was eluted by applying 3.0 ml of sterile H₂O followed by centrifugation at 3000×gravity for five minutes. Each binding column was then rinsed using 20 ml of sterile H₂O centrifuging at 3000×gravity for five minutes.

Flow-throughs from the first binding step for each of the samples were then reloaded into their respective binding columns and the binding, wash, drying, elution, and column rinsing procedures were repeated in identical fashion. Two subsequent binding and elutions followed, resulting in a total of four-3.0 ml elutions representing each sample. This was done to ensure that differences in overall yield between the conditions were not simply a reflection of the binding efficiency or column capacity. Yield estimations were done by spectrophotometry, and resultant yields for the four-elution sets were combined to reflect the total yield of plasmid DNA. The following results were obtained:

| O.D.₆₀₀ cell mass | Plasmid yield in μg | Lysate Volume |
|---|---|---|
| Processed | Bubbles added | No bubbles added |
| 500 | 1933 | 2175 |
| 1000 | 4108 | 4108 |
| 1250 | 4833 | 4108 |
| 1500 | 4350 | 3383 |
| 2000 | 4350 | 4108 |

### Example 12

### Making Buoyant Networks of PVDF (Polyvinylidene Difluoride) Particles Covered with SiO₂ by Column Synthesis Method

Two solutions were prepared for later use in the procedure: (1) “SiO₂-KOH” was made to a final formulation of 9.0% SiO₂ in 5.8% KOH and (2) 1.0 N HCl.

gm of Hylar 461 PVDF particles (Solvay Solexis, Brussels, Belgium) were weighed in a clearing column (see Example 7), and 7 ml of SiO₂-KOH was added, and the contents mixed thoroughly. The suspension was added to a Promega (Madison, Wis., USA) catalog #A246B PureYield™ Clearing Column placed in a 50 ml plastic tube, and the solution was allowed to drip through the clearing column for 20 minutes at 1×gravity.

10 ml of 1 N HCl was added to the column. The column was allowed to drip at 1×gravity for 5 minutes, then the pH of the ending flow-through solution on the bottom of the column was tested, and found to be about pH 2 by pH indicator paper. The particles were transferred from the column into a 50 ml plastic tube using three transfers of 15 ml each of water, in which the particles were mixed using a 10 ml plastic pipet and transferred to the 50 ml tube. After 10 minutes at 1×gravity, the solution below the buoyant network particles was removed using a 10 ml pipette. 30 ml of 200 mM KOAc, pH 4.8 was added, and the contents mixed. After 10 minutes at 1×gravity, the bottom solution was removed. The pH of the removed solution was tested by pH paper and found to be about pH 4.8. 30 ml of water was added and the contents mixed. After 10 minutes at 1×gravity, the solution below was removed. The buoyant networks of particles were resuspended in 5 ml of water. After 30 minutes at 1×gravity, the solution was removed by pipetting, and the buoyant networks of particles were dried overnight at 20-22° C. and 1 atmosphere.

### Example 13

### Making Buoyant Networks of High Density Polyethylene (HDPE) Particles Covered with SiO₂ by Column Synthesis Method

Two solutions were prepared for later use in the procedure: (1) “SiO₂-KOH” was made to a final formulation of 9.0% SiO₂ in 5.8% KOH and (2) 1.0 N HCl.

3.0 gm of Inhance HD-1800 surface modified HDPE PD-045.01-1 (Fluoro-Seal, Houston, Tex.) were weighed in a 50 ml plastic tube, and 4 ml of SiO₂-KOH was added, and the contents mixed thoroughly. The suspension was added to a Promega (Madison, Wis., USA) catalog #A246B PureYield™ Clearing Column placed in a 50 ml plastic tube, and the solution was allowed to drip through the clearing column for 40 minutes at 1×gravity.

10 ml of 1 N HCl was added to the column. The column was allowed to drip at 1×gravity for 60 minutes, then the pH of the ending flow-through solution on the bottom of the column was tested, and found to be about pH 2 by pH indicator paper. 10 ml of 200 mM KOAc, pH 4.8 was added. After 30 minutes at 1×gravity, the bottom solution was removed. The pH of the solution at the bottom of the column was tested by pH paper and found to be about pH 4.8. 10 ml of water was added and the column was allowed to drip for 40 minutes at 1×gravity. 10 ml of water was added and the column was allowed to drip for another 90 minutes at 1×gravity. The buoyant HDPE-silica networks of particles were removed to a clean 50 ml tube, and the remaining solution was removed using a pipette. The buoyant HDPE-silica networks of particles were dried overnight at 20-22° C. and 1 atmosphere.

### Example 14

### Clearing Lysates Using PVDF, Networks of PVDF-silica, HDPE, and Networks of HDPE-silica Buoyant Particles

50 ml of Luria Broth (LB-Miller) bacterial plasmid culture JM109 (pTMV266) (a low copy chloramphenicol resistance & tobacco mosaic virus sequence containing plasmid) was centrifuged into sixteen 50 ml conical screw cap centrifuge tubes. This was repeated for a total of six repetitions per tube. The result was 16 tubes, each with 300 ml of bacterial culture (A600 of 2.2 per ml) pelleted per tube. The tubes were labeled as “tubes A 660 ODs” and were frozen at −20° C. for later plasmid DNA extraction.

50 ml of Luria Broth (LB-Miller) bacterial plasmid culture JM109 (pTMV266) was centrifuged into sixteen 50 ml conical screw cap centrifuge tubes. This was repeated for a total of 5 repetitions per tube. An additional 10 ml per tube was then centrifuged. The result was 16 tubes, each with 260 ml of bacterial culture (A600 of 1.9 per ml) pelleted per tube. The tubes were labeled as “tubes B 490 ODs” and were frozen at −20° C. for later plasmid DNA extraction.

Plasmid purification was performed using Promega's (Madison, Wis.) A2495 plasmid midi-plasmid purification system, with the following solution compositions:
Cell Resuspension Solution: 50 mM Tris, 10 mM EDTA, 100 μg/ml RNase A;
Cell Lysis Solution: 0.2 M Sodium Hydroxide, 1% SDS;
Neutralization Solution: 4.09 M Guanidine Hydrochloride, 759 mM potassium acetate, 2.12 M glacial acetic acid;
Column Wash: 162.8 mM Potassium Acetate, 22.6 mM Tris, 0.109 mM EDTA. To 320 ml add 170 ml of 95% ethanol;
A246B PureYield™ Clearing Columns were used for lysate clearing; and
A245B PureYield™ Binding Columns were used for purification of plasmid DNA.

To 14 tubes of “tubes B” cell pellets, above, 4.0 ml of Cell Resuspension solution was added and mixed by vigorous vortexing. The resuspended bacterial cells were then transferred to each of 14 of “tubes A”. Each tube was vigorously vortexed to resuspend the bacterial cells. 1.0 ml of Cell Resuspension solution was added to each of the “tubes B”, the tubes were rinsed, and the resuspended cells added to their respective “tubes A” counterpart to provide a combined cell mass of 1150 A600 optical density units in 5 ml of Resuspension Solution. Tubes B were discarded.

Then 5 ml of Cell Lysis solution was added to tubes A and mixed gently. Then 9 ml of Neutralization Solution were added per tube, and gently mixed.

To each of the tubes A above, the following were added:
Tubes 1 and 2: no buoyant particles were added;
Tubes 3 and 4: 0.7 gm PVDF (see Example 12) were added;
Tubes 5 and 6: 0.5 gm PVDF networks of buoyant particles (see Example 12) were added;
Tubes 7 and 8: 0.7 gm HDPE (see Example 13) were added;
Tubes 9 and 10: 0.7 gm HDPE networks of buoyant particles (see Example 13) were added;
Tubes 11 and 12: 0.7 gm Scotchlite™ H50 hydrolyzed (see Example 5) glass bubbles were added; and
Tubes 13 and 14: samples were centrifuged at 2200×gravity for 10 minutes, liquid was removed by pipette aspiration (from pockets within debris).

Each tube was mixed and added to an A246B PureYield™ Clearing Column, each of which was contained in a 50 ml tube. The solutions were allowed to sit in the columns for 2 minutes, then the tubes were centrifuged for 10 minutes at 2200×gravity, and the flow-through solutions captured in the 50 ml tubes. The volume contents per 50 ml tube after filtration/centrifugation were as shown in the table of results below.

The contents of each tube were added to an A245B PureYield™ Binding Column, then the tubes were centrifuged for 10 minutes at 2200×gravity. The flow-throughs were discarded, and the binding columns washed with 5 ml of Endotoxin Wash per tube, and centrifuged at 2200×gravity for 10 minutes. The wash flow-throughs were discarded, and the columns washed with 20 ml of column wash per tube, and centrifuged at 2200×gravity for 10 minutes. The columns were transferred to clean 50 ml tubes and eluted with 800 μl of nuclease free water. After 5 minutes at 21° C., tubes were centrifuged at 2200×gravity for 5 minutes, and a second elution of 800 μl nuclease free water was added per column. After 5 minutes at 21° C., the columns were centrifuged at 2200×gravity for 5 minutes, thus combining elutions 1 and 2. The sample DNA was analyzed and frozen at −20° C. The results are shown in the table below:

| | ml Volume of Lysate | |
|---|---|---|
| Samples | Flow-through | μg by PicoGreen |
| No particles A | 2.3 | 25.7 |
| No particles B | 1.8 | 34.1 |
| PVDF A | 1.4 | 12.9 |
| PVDF B | 1.4 | 14.6 |
| Network PVDF A | 1.2 | 18.0 |
| Network PVDF B | 1.4 | 19.3 |
| HDPE A | 9.8 | 37.9 |
| HDPE B | 9.9 | 36.7 |
| Network HDPE A | 9.7 | 16.8 |
| Network HDPE B | 5.9 | 21.4 |
| H50 hydrolyzed A | 6.3 | 52.7 |
| H50 hydrolyzed B | 9.0 | 55.1 |
| Centrifuged A | 8.4 | 19.8 |
| Centrifuged B | 7.0 | 18.4 |

### Example 15

### Clearing Debris and Non-target DNA Using S60 and Networks of S60 Particles, Prior to Purification of a Non-nucleic Acid Target Molecule

Example 5 above shows the DNA binding properties of a variety of buoyant particles. As can be seen in the results, the S60/10,000 Scotchlite™ bubbles and the (not silanized) networks of particles showed a greater capacity for DNA binding than silanized particles or silanized networks of particles, or the H50/10,000 Scotchlite™ bubbles. In this example, the higher binding capacity particles (S60 and S60 network buoyant particles) were used to both clear the lysate of debris, and to remove plasmid DNA that might interfere with the subsequent purification of the desired, non-nucleic acid, target product. While the silanized particles were generally preferred for purification of target nucleic acids (as shown in Examples 6 and 7, for example), the S60 buoyant particles and the S60 networks of buoyant particles (as used in this example) showed preferred properties for purifying non-nucleic acid targets (where the non-target DNA may undesirably copurify with the target molecule(s)).

50 ml of Luria Broth (LB-Miller) bacterial plasmid culture JM109 (pMGFP) was centrifuged into sixteen 50 ml conical screw cap centrifuge tubes. This was repeated for a total of four repetitions per tube. An additional 25 ml per tube was centrifuged. The result was 16 tubes, each with 225 ml of bacterial culture pelleted per tube. The tubes were labeled as “tubes A” and were frozen at −20° C. for later plasmid DNA extraction.

50 ml of Luria Broth (LB-Miller) bacterial plasmid culture JM109 (pMGFP) was centrifuged into sixteen 50 ml conical screw cap centrifuge tubes. This was repeated for a total of four repetitions per tube. The result was 16 tubes, each with 200 ml of bacterial culture pelleted per tube. The tubes were labeled as “tubes B” and were frozen at −20° C. for later plasmid DNA extraction.

Plasmid purification was performed using Promega's (Madison, Wis.) A2495 plasmid midi-plasmid purification system, with the following solution compositions:
Cell Resuspension Solution: 50 mM Tris, 10 mM EDTA, 100 μg/ml RNase A;
Cell Lysis Solution: 0.2 M Sodium Hydroxide, 1% SDS;
Neutralization Solution: 4.09 M Guanidine Hydrochloride, 759 mM potassium acetate, 2.12 M glacial acetic acid;
Column Wash: 162.8 mM Potassium Acetate, 22.6 mM Tris, 0.109 mM EDTA. To 320 ml add 170 ml of 95% ethanol;
A246B PureYield™ Clearing Columns were used for lysate clearing; and
A245B PureYield™ Binding Columns were used for purification of plasmid DNA.

To 8 tubes of “tubes B” above cell pellets, 4.0 ml of Cell Resuspension solution was added and mixed by vigorous vortexing. The resuspended bacterial cells were then transferred to each of 8 tubes of JM109 (phmGFP) 225 ml pellets (tubes A). Each tube was vigorously vortexed to resuspend the bacterial cells. 1.0 ml of Cell Resuspension solution was added to each of the “tubes B”, the tubes were rinsed, and the resuspended cells added to their respective “tubes A” counterpart to provide a combined cell mass of 425 ml of bacterial culture in 5 ml of Resuspension Solution. Tubes B were discarded.

To each of the tubes A above, the following were added:
Tubes 1 and 2: no buoyant particles were added;
Tubes 3 and 4: 0.5 gm of S60/10,000 Scotchlite™ bubbles were added;
Tubes 5 and 6: 1.0 gm of S60/10,000 Scotchlite™ bubbles were added; and
Tubes 7 and 8: 0.5 gm of network-S60 particles were added.

Then 5 ml of Cell Lysis solution was added to tubes A and mixed gently. Then 9 ml of Neutralization Solution were added per tube, and gently mixed. The contents of each tube were added to an A246B PureYield™ Clearing Column each of which was contained in a 50 ml conical bottom tube. The solutions were allowed to sit in the columns for 2 minutes, then the tubes were centrifuged for 10 minutes at 2000×gravity, and the flow-through solutions captured in 50 ml conical tubes. The volume contents per 50 ml tube were:
Tubes 1, 2=12.5 ml, 12.5 ml (both tubes clogged);
Tubes 3, 4=15.5 ml, 15.5 ml;
Tubes 5, 6=14.5 ml, 14.5 ml; and
Tubes 7, 8=15 ml, 15 ml. None of tubes 3-8 clogged.

The contents of each tube were added to an A245B PureYield™ Binding Column, then the tubes were centrifuged for 10 minutes at 2000×gravity. The flow-through was saved for later use. Each column was washed with 10 ml of column wash (above), and then the tubes were centrifuged for 10 minutes at 2000×gravity.

Plasmid DNA was eluted by addition of 5 ml of water, then the columns were allowed to drip for 10 minutes, followed by a second elution of 5 ml of water. The columns were then centrifuged 5 minutes at 2000×gravity. The binding columns were then reused, by applying the previously saved lysate flow-through to their respective binding columns. The columns were washed with column wash, as described above, and the DNA eluted as above. The results are shown in the following table:

| | ml lysate | PicoGreen | μg (1st + 2nd) |
|---|---|---|---|
| Sample | flow-through | μg | elution |
| No buoy A 1^{st} elution | 12.5 | 123 | 258 |
| No buoy B 1^{st} elution | 12.5 | 109 | 260 |
| 0.5 gm S60 A 1^{st} elution | 15.5 | 23 | 50 |
| 0.5 gm S60 B 1^{st} elution | 15.5 | 89 | 198 |
| No buoy A 2^{nd} elution | | 135 | |
| No buoy B 2^{nd} elution | | 151 | |
| 0.5 gm S60 A 2^{nd} elution | | 27 | |
| 0.5 gm S60 B 2^{nd} elution | | 109 | |
| 1.0 gm S60 A 1^{st} elution | 14.5 | 88 | 119 |
| 1.0 gm S60 B 1^{st} elution | 14.5 | 81 | 116 |
| Network A 1^{st} elution | 15.0 | 140 | 223 |
| Network B 1^{st} elution | 15.0 | 139 | 238 |
| 1.0 gm S60 A 2nd elution | | 31 | |
| 1.0 gm S60 B 2nd elution | | 35 | |
| Network A 2nd elution | | 83 | |
| Network B 2nd elution | | 99 | |

In cases where nucleic acid is not the desired target for purification, buoyant particles can be used to remove debris as well as undesired nucleic acids, prior to the purification of the desired non-nucleic acid product.

### Example 16

### Purification of DNA From Plant Material Using H50 Scotchlite™ Glass Bubbles, PVDF Buoyant Particles, HDPE Buoyant Particles, Networks of S60 Scotchlite™ Particles (and No Particle and Centrifugation Controls)

Using Promega's Wizard® Magnetic DNA Purification System for Food (Cat #FF3751, Madison, Wis.), DNA was purified from 3.5 gm of Gardenburger® Vegie medley-vegan burger patty (Gardenburger Authentic Foods Company, Clearwater, Utah). Each of the samples listed below was processed in a plastic 50 ml screw-cap tube. To each tube was added: 3.5 gm of Gardenburger material (containing corn, soy, oats, wheat, carrot). Then 50 μl RNase A was added, followed by 5 ml of Buffer A, and the contents mixed. Then 2.5 ml of Buffer B was added, the contents mixed and incubated at 21° C. for 10 minutes. Then 7.0 ml of Precipitation Solution was added, and the contents mixed. For tubes in which particles were added, 0.5 gm of the respective particles were added per tube. The contents of each sample was mixed, and poured into their respective PureYield Clearing columns, each contained in a 50 ml plastic screw-cap tube (as described in Example 15). After waiting 1 minute, the clearing columns in tubes were spun at 2000×gravity for 30 minutes. The liquid volumes of cleared lysate present in the bottom of the tubes was measured, as shown in the table below.

It was necessary to centrifuge the “centrifugation controls” a second time to reduce the amount of particulate present in the samples. After removal of the clearing column from each tube, 400 μl of MagneSil™ paramagnetic particles were added per tube. After mixing, 0.8 volumes of isopropanol was added (volume in table below plus 400 μl from MagneSil™ addition), and the tubes mixed after 2, 5 and 10 minutes at 21° C. The tubes were placed on a magnetic stand for 1 minute, and the solution was then discarded (leaving the MagneSil™ paramagnetic particles). After removal from the magnetic stand, 5 ml of Buffer B was added per tube, and mixed. The tubes were placed back on the magnetic stand, and after 1 minute, the solution was discarded. After removal from the magnetic stand, 15 ml of 70% (vol/vol) ethanol/water was added as a wash (per tube). The tubes were placed on the magnetic stand, and after 1 minute, the solution was discarded. The 70% ethanol wash steps were repeated twice, for a total of three washes. After the final wash was discarded, the tubes were air dried at 21° C. for 45 minutes while on the magnetic rack. The tubes were removed from the magnetic rack, and the MagneSil™ paramagnetic particles were eluted with 500 μl of nuclease free water for 15 minutes at 21° C. The tubes were placed back on the magnetic stand, and after 1 minute, the solution containing eluted DNA was removed from each of the tubes and placed into their respective 1.5 ml plastic tubes. Total ng of DNA per sample was determined using PicoGreen™ (Invitrogen, Carlsbad, Calif.). The results are shown below:

| Sample | lysate ml volume cleared | ng DNA by PicoGreen |
|---|---|---|
| No buoyant particles A | 3.3 | 590 |
| No buoyant particles B | 2.9 | 633 |
| H50 hydrolyzed A | 12.9 | 1036 |
| H50 hydrolyzed B | 8.8 | 823 |
| PVDF A | 13.0 | 932 |
| PVDF B | 2.6 | 452 |
| HDPE A | 7.0 | 978 |
| HDPE B | 9.4 | 1061 |
| Network S60 A | 9.0 | 968 |
| Network S60 B | 15.0 | 1177 |
| Twice centrifuged A | 12.5 | 1015 |
| Twice centrifuged B | 12.3 | 1042 |

We claim:

1. A network of buoyant particles for clearing lysates of biological material, the network comprising:
two or more buoyant particles covalently linked together, wherein the network ranges in size from approximately 30 microns to approximately one centimeter along the network's longest dimension.

2. The network according to claim 1, wherein the buoyant particles have a silica or a silica-containing surface.

3. The network according to claim 1, wherein the network has a density less than about 1.2 g/cm³.

4. The network according to claim 1, wherein the network ranges in size from approximately 100 microns to approximately 1 mm along the network's longest dimension.

5. The network according to claim 4, wherein the network ranges in size from approximately 100 microns to approximately 500 microns along the network's longest dimension.

6. A method of making a network of buoyant particles for clearing lysates of biological material, the method comprising the steps of:
(a) placing at least two buoyant particles in an alkaline solution containing SiO₂, and
(b) adding acid to the solution so that the SiO₂ condenses, covalently linking the at least two buoyant particles together to form the network of buoyant particles.

7. The method according to claim 6, wherein the at least two buoyant particles have a silica or a silica-containing surface.

8. The method according to claim 7, wherein the network ranges in size from approximately 30 microns to approximately 1 mm along the network's longest dimension.

9. The method according to claim 8, wherein the network has a size ranging from approximately 100 microns to approximately 500 microns along the network's longest dimension.

10. The method according to claim 6, wherein the network has a density less than about 1.2 g/cm³.

11. A method of making a network of buoyant particles for clearing lysates of biological material, the method comprising the steps of:
(a) placing at least two buoyant particles having a silica or a silica-containing surface in an alkaline solution, and
(b) combining the result of step (a) with a salt plus acid solution.

12. The method according to claim 11, wherein the network ranges in size from approximately 30 microns to approximately 1 mm along the network's longest dimension.

13. The method according to claim 12, wherein the network has a size ranging from approximately 100 microns to approximately 500 microns along the network's longest dimension.

14. The method according to claim 11, wherein the network has a density less than about 1.2 g/cm³.

15. A method of isolating target biological material, the method comprising the steps of:
(a) adding a buoyant particle, a network of buoyant particles, or mixtures thereof to a sample of biological material;
(b) adding a binding solution;
(c) performing cell lysis; and
(d) separating target biological material and non-target biological material by gravity, centrifugation vacuum filtration or positive pressure filtration,
wherein the binding solution is added at a concentration sufficient to promote selective adsorption of the target or non-target biological material to the buoyant particle, the network of buoyant particles, or mixtures thereof.

16. The method of claim 15, wherein the sample of biological material is at least one of bacteria, plant tissue, animal tissue or animal body fluids.

17. The method of claim 15, further comprising a step of purifying the target biological material.

18. The method according to claim 15, wherein the binding solution contains at least one of a chaotrope and an alcohol.

19. The method according to claim 15, further comprising a step of heating the solution after performing cell lysis.

20. The method according to claim 15, wherein the target biological material is DNA and the non-target biological material is RNA.

21. The method according to claim 15, wherein the target biological material is RNA and the non-target biological material is DNA.

22. The method according to claim 15, wherein the target biological material is plasmid DNA and the non-target biological material is genomic DNA.

23. A method of isolating target biological material, the method comprising the steps of:
(a) combining a buoyant particle, a network of buoyant particles, or mixtures thereof with lysed biological material;
(b) adding a binding solution; and
(c) separating the biological material by gravity, centrifugation, vacuum filtration or positive pressure filtration,
wherein the binding solution is added at a concentration sufficient to promote selective adsorption of the target or non-target biological material to the buoyant particle, the network of buoyant particles, or mixtures thereof.

24. The method of claim 23, wherein the biological material is at least one of bacteria, plant tissue, animal tissue or animal body fluids.

25. The method according to claim 23, further comprising a step of purifying the target biological material.

26. The method according to claim 23, wherein the binding solution contains at least one of a chaotrope and an alcohol.

27. The method according to claim 23, further comprising a step of heating the solution prior to the separation by gravity or centrifugation.

28. The method according to claim 23, wherein the target biological material is DNA and the non-target biological material is RNA.

29. The method according to claim 23, wherein the target biological material is RNA and the non-target biological material is DNA.

30. The method according to claim 23, wherein the target biological material is plasmid DNA and the non-target biological material is genomic DNA.

31. A method of isolating target biological material, the method comprising the steps of:
(a) combining lysed biological material with;
(b) a binding solution containing a buoyant particle, a network of buoyant particles, or mixtures thereof; and
(c) separating the biological material by gravity, centrifugation, vacuum filtration or positive pressure filtration,
wherein the binding solution is added at a concentration sufficient to promote selective adsorption of the target or non-target biological material to the buoyant particle, the network of buoyant particles, or mixtures thereof.

32. The method of claim 31, wherein the biological material is at least one of bacteria, plant tissue, animal tissue or animal body fluids.

33. The method according to claim 31, further comprising a step of purifying the target biological material.

34. The method according to claim 31, wherein the binding solution contains at least one of a chaotrope and an alcohol.

35. The method according to claim 31, further comprising a step of heating the solution prior to the separation by gravity, centrifugation, vacuum filtration or positive pressure filtration.

36. The method according to claim 31, wherein the target biological material is DNA and the non-target biological material is RNA.

37. The method according to claim 31, wherein the target biological material is RNA and the non-target biological material is DNA.

38. The method according to claim 31, wherein the target biological material is plasmid DNA and the non-target biological material is genomic DNA.

39. A kit comprising a container containing a lysis solution and at least one member selected from the group consisting of a buoyant particle, a network of buoyant particles, and a buoyant particle and a network of buoyant particles.

40. The kit according to claim 39, further comprising a clearing column.

41. A kit for clearing lysates of biological material, the kit comprising:
a container containing a lysis solution; and
at least one other container,
wherein the at least one other container is selected from the group consisting of a container containing at least one buoyant particle, a container containing at least one network of buoyant particles, and a container containing at least one buoyant particle and at least one network of buoyant particles.

42. The kit according to claim 41, further comprising a clearing column.
